(19) United States
(12) Patent Application Publication
(10) Pub. No.: US 2010/0186764 A1
(43) Pub. Date: Jul. 29, 2010

(54) COSMETIC COMPOSITION COMPRISING AT LEAST ONE BRANCHED SULPHONIC POLYESTER AND AT LEAST ONE (METH)ACRYLIC THICKENER AND METHODS OF USING IN HAIR STYLING

(76) Inventors: Dorothée Pasquet, Bois Colombes (FR); Nadia Smail, Verneuil Sur Seine (FR); Jonathan Gawtrey, Boulogne (FR); Ceçile Bebot, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/640,608

(22) Filed: Dec. 17, 2009

Related U.S. Application Data
(60) Provisional application No. 61/146,412, filed on Jan. 22, 2009.

(30) Foreign Application Priority Data
Dec. 17, 2008 (FR) 0858673

(51) Int. Cl. A45D 7/04; A61K 8/81

(52) U.S. Cl. 132/203; 424/70.16; 424/70.13

(57) ABSTRACT

The present application relates to cosmetic compositions comprising, in a cosmetically acceptable medium: (i) at least one branched sulphonic polyester; (ii) at least one (meth)acrylic thickening polymer and (iii) at least one surfactant chosen from anionic, non-ionic, amphoteric, and cationic surfactants. The present disclosure also relates to methods of using the disclosed compositions for styling or shaping keratin materials.

This application claims benefit of U.S. Provisional Application No. 61/146,412, filed Jan. 22, 2009. This application also claims benefit of priority under 35 U.S.C. §119 to French Patent Application No. 0858673, filed Dec. 17, 2008.

The present disclosure relates to novel cosmetic compositions comprising at least one branched sulphonic polyester, at least one (meth)acrylic thickening polymer and at least one surfactant chosen from anionic, non-ionic, amphoteric, and cationic surfactants, and also to the uses of these compositions for, styling or shaping keratin materials.

The present disclosure also relates to a method of styling keratin materials using these compositions.

The use of branched sulphonic polyesters in hair styling and fixing compositions is known and described, for example, in European Patent Application Publication No. EP 0 966 946, International Patent Application Publication Nos. WO 98/38969 and WO 99/63955.

Nevertheless, the use of branched sulphonic polyesters is not without drawbacks:
the use of these polyesters in hair lacquers containing large quantities of alcohol generally gives good styling properties but does not make it possible to obtain sufficient lacquering power;
the application of these polyesters in the form of lacquers with a high alcohol content gives the hair, after brushing, a dry feel; this undesirable phenomenon is noticeable for dyed hair, and
these polyesters are generally in semi-solid form, and this property often makes their use difficult, for instance, it is often difficult to ensure a homogeneous distribution of these polyesters over all the hair to be treated.

Unexpectedly and advantageously, Applicants have found that the use of at least one branched sulphonic polyester with at least one (meth)acrylic thickening polymer and at least one surfactant makes it possible to overcome the aforementioned drawbacks.

One aspect of the present disclosure is thus a cosmetic composition comprising at least one branched sulphonic polyester, at least one (meth)acrylic thickening polymer, and at least one surfactant chosen from anionic, non-ionic, amphoteric, and cationic surfactants.

The term “(meth)acrylic thickening polymers” is understood within the meaning of the present application to mean non-fixing thickening (meth)acrylic polymers.

The compositions obtained can be in the form of gels, mousses, sprays, creams or pastes.

The compositions according to the present disclosure can be easy to prepare and to apply. For example, they remain localized, without runs, at the point of application. The compositions according to the present disclosure may be applied without a reduction in viscosity over time.

Moreover, the compositions according to the present disclosure make it possible to give the hairstyle a natural and long-lasting form retention.

Another aspect of the present disclosure is a method for styling keratin materials, for example, human keratin materials such as the hair, which uses the compositions according to the present disclosure.

Another aspect of the present disclosure is the uses of the compositions according to the present disclosure, for instance, for styling and shaping of keratin materials, for example, human keratin materials such as the hair.

Other features, aspects, subjects and advantages of the present disclosure will appear even more clearly on reading the description and examples that follow.

The term “(meth)acrylic” within the meaning of the present application is understood to mean “acrylic or methacrylic”.

The at least one branched sulphonic polyesters used in the compositions of the present disclosure are known in the prior art. Their structure and synthesis are described in International Patent Application Publication Nos. WO 95/18191, WO 97/08261 and WO 97/20899.

In some embodiments, the at least one branched sulphonic polyester is obtained by polycondensation of:

(a) at least one dicarboxylic acid that does not bear a sulphonic functional group,

(b) at least one diol or a mixture comprising at least one dial and at least one diamine,

(c) at least one monomer comprising two identical or different reactive functional groups chosen from hydroxyl, amino, and carboxyl groups, and at least one sulphonic functional group, and

(d) at least one monomer comprising at least three identical or different reactive functional groups chosen from hydroxyl, amino, and carboxyl groups.

The dicarboxylic acids that form the units (a) may be chosen from aliphatic dicarboxylic acids, alicyclic dicarboxylic acids, aromatic dicarboxylic acids, and mixtures of such acids.

Mention may be made, by way of non-limiting example, of 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid and isophthalic acid and mixtures of such acids.

The at least one diol that form the units (b) are chosen, for example, from alkanediols and polyalkylenediols, and mention may be made, by way of non-limiting example, of ethylene glycol, propylene glycol, diethylene glycol, triethylene glycol and polypropylene glycol.

The diamines capable of forming one part of the units (b) are chosen from, by way of non-limiting example, alkanediamines and polyoxyalkylene diamines.

The expression “sulphonic functional group” of the units (c) encompasses both the sulphonic acid functional group (—SO3H) and the corresponding salified functional groups obtained by neutralization of the sulphonic acid functional group with a base, for example, an alkali metal hydroxide.

The sulphonic functional groups can be, for example, in the form which is neutralized by an organic or inorganic base.

The units (c) are derived, for example, from dicarboxylic acids, dicarboxylic acid esters, glycols and hydroxy acids, all bearing at least one sulphonic group, in acid and/or neutralized form, such as in neutralized form.

The units (c) bearing at least one sulphonic functional group may be present in an amount ranging from 2 to 15 mol % of the total amount of monomers.

The units (d) derived from multifunctional monomers may be present in an amount ranging from 0.1 to 40 mol % relative to the total amount of monomers.

The multifunctional monomers forming the units (d) are chosen, for example, from trimethylolethane, trimethylolpropane, glycerol, pentaerythritol, sorbitol, trimellitic anhydride, erythritol, threitol, dipentaerythritol, pyromellitic dianhydride, and dimethylpropionic acid.

The at least one branched sulphonic polyester may comprise, in addition to the four types of units (a) to (d) described above, units (e) derived from monomers comprising two different reactive functional groups, chosen from, for example, hydroxy carboxylic acids and amino carboxylic acids or mixtures thereof.

These units (e) may be present in an amount up to 40 mol % of the total amount of monomers (a), (b), (c), (d) and (e).

Further as a non-limiting example, the at least one branched sulphonic polymer used in the present disclosure can be obtained from a mixture of monomers in which the number of equivalents of carboxylic acid functional groups is substantially equal to the number of equivalents of hydroxyl functional groups and of amino functional groups which may be present.

The at least one branched sulphonic polymer used in the styling compositions of the present disclosure are known and sold, for example, by Eastman. Non-limiting mention may be made of the product sold under the name AQ 1350® by Eastman.

The composition according to the present disclosure may comprise at least one branched sulphonic polyesters in an amount ranging from 0.2 to 15%, for example, from 0.5 to 10% such as from 1 to 10% by weight, relative to the total weight of the composition.

The composition according to the present disclosure also comprises at least one (meth)acrylic thickening polymer.

The term “acrylic polymer” as used herein refers to polymers that result from the polymerization of at least one monomer of structure:

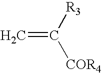

R₃ is chosen from a hydrogen atom and linear or branched C₁-C₄ alkyl radicals,

R₄ is chosen from a hydrogen atom, linear or branched C₁-C₄ alkyl radicals, NR₅R₆ radicals, and linear or branched C₁-C₃₀ alkoxy radicals, optionally substituted with at least one hydroxyl radical or with a quaternary ammonium radical,

R₅ and R₆ are chosen from a hydrogen atom and optionally oxyalkylenated C₁-C₃₀ alkyl radicals, the alkyl radicals optionally comprising a sulphonic group.

As a non-limiting example, R₃ is chosen from a hydrogen atom and a methyl radical.

For the purposes of the present disclosure, the term “thickening polymer” refers to a polymer capable, by its presence, of increasing the viscosity of the medium by at least 50 centipoise at 25° C. and at a shear rate of 1 s⁻¹. For instance, the thickening polymer has, at 1% in water or in a 50/50 water/alcohol mixture by weight at 25° C., a viscosity of greater than 100 centipoise at a shear rate of 1 s⁻¹. These viscosities may be measured with viscometers or rheometers with cone-plate geometry.

In certain embodiments, the acrylic thickening polymers are chosen from:

(a) acrylic associative thickeners;

(b) crosslinked acrylic acid homopolymers;

(c) crosslinked copolymers of (meth)acrylic acid and of a (C₁-C₆)alkyl acrylate;

(d) non-ionic homopolymers and copolymers containing ethylenically unsaturated monomers of ester and/or amide type;

(e) ammonium acrylate homopolymers;

(f) copolymers of ammonium acrylate and of acrylamide;

(g) (meth)acrylamido(C₁-C₄)alkylsulphonic acid homopolymers and copolymers; and

(h) crosslinked methacryloyl(C₁-C₄)alkyltri(C₁-C₄)alkylammonium homopolymers and copolymers.

According to the present disclosure, the term “associative thickener” refers to an amphiphilic thickener comprising both hydrophilic units and hydrophobic units, for example, comprising at least one C₈-C₃₀ fatty chain and at least one hydrophilic unit.

Acrylic associative thickeners that may be used according to the present disclosure are acrylic associative polymers chosen from:

(i) non-ionic amphiphilic polymers comprising at least one fatty chain and at least one hydrophilic unit;

(ii) anionic amphiphilic polymers comprising at least one hydrophilic unit and at least one fatty-chain unit;

(iii) cationic amphiphilic polymers comprising at least one hydrophilic unit and at least one fatty-chain unit;

(iv) amphoteric amphiphilic polymers comprising at least one hydrophilic unit and at least one fatty-chain unit;
the fatty chains containing from 10 to 30 carbon atoms.

In some embodiments, the acrylic non-ionic amphiphilic polymers in (i) comprising at least one fatty chain and at least one hydrophilic unit are chosen from:

(1) copolymers of C₁-C₆ alkyl methacrylates or acrylates and of amphiphilic monomers comprising at least one fatty chain (for example oxyethylenated (C₈-C₂₂)alkyl acrylates), for instance the oxyethylenated methyl methacrylate/stearyl acrylate copolymer sold by the company Goldschmidt under the name ANTIL 208;

(2) copolymers of hydrophilic methacrylates or acrylates and of hydrophobic monomers comprising at least one fatty chain (for example (C₈-C₂₂)alkyl (meth)acrylates), for instance polyethylene glycol methacrylate/lauryl methacrylate copolymer.

In some embodiments, the acrylic anionic amphiphilic polymers in (ii) can be chosen from those comprising at least one hydrophilic unit of unsaturated olefinic carboxylic acid type, and at least one hydrophobic unit of the type such as a (C₁₀-C₃₀) alkyl ester of an unsaturated carboxylic acid. Further as a non-limiting example, they are chosen from those having the hydrophilic unit of unsaturated olefinic carboxylic acid type corresponds to the monomer of formula (I) below:

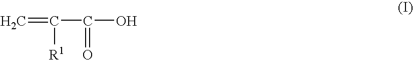

wherein formula R¹ is chosen from H, CH₃, and C₂H₅ (i.e., acrylic acid, methacrylic acid or ethacrylic acid units), and wherein the hydrophobic unit of the type (C₁₀-C₃₀) alkyl ester of an unsaturated carboxylic acid corresponds to the monomer of formula (II) below:

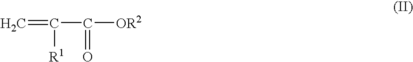

wherein formula R¹ is chosen from H, CH₃, C₂H₅ (i.e., acrylate, methacrylate or ethacrylate units), for instance, R¹ is H (acrylate units) or CH₃ (methacrylate units), R² is chose from C₁₀-C₃₀ radicals such as a C₁₂-C₂₂ alkyl radical.

(C₁₀-C₃₀)Alkyl esters of unsaturated carboxylic acids in accordance with the present disclosure comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate, and dodecyl methacrylate.

Anionic amphiphilic polymers of this type are disclosed and prepared, for example, according to the U.S. Pat. No. 3,915,921 and U.S. Pat. No. 4,509,949.

The anionic amphiphilic polymers that can be used in the context of the present disclosure may also be chosen from polymers formed from a mixture of monomers comprising:

(i) acrylic acid and at least one ester of formula (III) below:

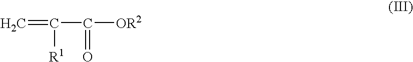

wherein R¹ is chosen from H and CH₃, R² is chosen from alkyl radicals containing from 12 to 22 carbon atoms; and a crosslinking agent, such as those constituted of from 95% to 60% by weight of acrylic acid (hydrophilic unit), from 4% to 40% by weight of C₁₀-C₃₀ alkyl acrylate (hydrophobic unit), and from 0% to 6% by weight of crosslinking polymerizable monomer; or further as an example, from 98% to 96% by weight of acrylic acid (hydrophilic unit), from 1% to 4% by weight of C₁₀-C₃₀ alkyl acrylate (hydrophobic unit), and from 0.1% to 0.6% by weight of crosslinking polymerizable monomer; and

(ii) essentially acrylic acid and lauryl methacrylate, such as the product formed from 66% by weight of acrylic acid and 34% by weight of lauryl methacrylate.

In one embodiment, the crosslinking agent is a monomer containing a group

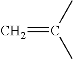

with at least one other polymerizable group whose unsaturated bonds are not conjugated relative to one another. Non-limiting mention may be made of polyallyl ethers such as, polyallyl sucrose and polyallyl pentaerythritol.

The polymers described above can be chosen from the products sold by the company Goodrich under the trade names PEMULEN TR1, PEMULEN TR2, CARBOPOL 1382, such as PEMULEN TR1, and the product sold by the company S.E.P.C. under the name COATEX SX.

As anionic amphiphilic fatty-chain polymers, non-limiting mention may also be made of the copolymer of methacrylic acid/methyl acrylate/dimethyl-meta-isopropenylbenzyl isocyanate of ethoxylated alcohol sold under the name VISCOPHOBE DB 1000 by the company Amerchol.

Other fatty-chain anionic amphiphilic polymers that may be mentioned include those comprising at least one acrylic monomer comprising sulphonic group(s), in free or partially or totally neutralized form and comprising at least one hydrophobic portion.

The hydrophobic portion present in the polymers of the present disclosure may contain from 8 to 22 carbon atoms, for example, from 8 to 18 carbon atoms such as from 12 to 18 carbon atoms.

The sulphonic polymers in accordance with the present disclosure can be partially or totally neutralized with an inorganic base (sodium hydroxide, potassium hydroxide or aqueous ammonia) or an organic base such as mono-, di- or triethanolamine, an aminomethylpropanediol, N-methylglucamine, basic amino acids, for instance arginine and lysine, and mixtures of these compounds.

The sulphonic amphiphilic polymers in accordance with the present disclosure may have a number-average molecular weight ranging from 1000 to 20 000 000 g/mol, such as ranging from 20 000 to 5 000 000, and further as an example, ranging from 100 000 to 1 500 000 g/mol.

The sulphonic amphiphilic polymers according to the present disclosure may or may not be crosslinked. For example, crosslinked amphiphilic polymers can be chosen.

When they are crosslinked, the crosslinking agents may be chosen from polyolefinically unsaturated compounds commonly used for the crosslinking of polymers obtained by free-radical polymerization. Mention may be made, for example, of divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric and/or vinylphosphonic acid derivatives, or mixtures of these compounds.

As a non-limiting example, methylenebisacrylamide, allyl methacrylate or trimethylolpropane triacrylate (TMPTA) can be used. The degree of crosslinking can range from 0.01 mol % to 10 mol % such as from 0.2 mol % to 2 mol % relative to the polymer.

The acrylic monomers containing sulphonic group(s) can be chosen from (meth)acrylamido(C₁-C₂₂)alkylsulphonic acids and N—(C₁-C₂₂)alkyl(meth)acrylamido(C₁-C₂₂)alkylsulphonic acids, for instance undecylacrylamidomethanesulphonic acid, and also partially or totally neutralized forms thereof.

(Meth)acrylamido(C₁-C₂₂)alkylsulphonic acids such as, for example, acrylamidomethanesuiphonic acid, acrylamidoethanesulphonic acid, acrylamidopropanesulphonic acid, 2-acrylamido-2-methylpropanesulphonic acid, methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid or 2-acrylamido-2,6-dimethyl-3-heptanesulphonic acid, and also partially or totally neutralized forms thereof, can be used.

2-Acrylamido-2-methylpropanesulphonic acid (AMPS), and also partially or totally neutralized forms thereof, may also be used.

The amphiphilic polymers in accordance with the present disclosure may be chosen from random amphiphilic AMPS polymers modified by reaction with a C₆-C₂₂ n-monoalkylamine or di-n-alkylamine, and such as those described in International Patent Application Publication No. WO 00/31154, the polymers described in this application form part of the content of the present description. These polymers may also contain other ethylenically unsaturated hydrophilic monomers chosen, for example, from (meth)acrylic acids, 13-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The polymers of the present disclosure may be chosen from amphiphilic copolymers of AMPS and of at least one ethylenically unsaturated hydrophobic monomer comprising at least one hydrophobic portion containing from 8 to 50 carbon atoms, for example, from 8 to 22 carbon atoms, and further as an example, from 8 to 18 carbon atoms such as 12 to 18 carbon atoms.

These same copolymers may also contain at least one ethylenically unsaturated monomers not comprising a fatty chain, such as (meth)acrylic acids, 13-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described in European Patent Application Publication No. EP-A-750 899, U.S. Pat. No. 5,089,578 and in the following publications from Yotaro Morishima:

“Self-assembling amphiphilic polyelectrolytes and their nanostructures—Chinese Journal of Polymer Science Vol. 18, No. 40, (2000), 323-336”;

“Micelle formation of random copolymers of sodium 2-(acrylamido)-2-methylpropanesulfonate and a non-ionic surfactant macromonomer in water as studied by fluorescence and dynamic light scattering—Macromolecules 2000, Vol. 33, No. 10-3694-3704”;

“Solution properties of micelle networks formed by non-ionic moieties covalently bound to a polyelectrolyte: salt effects on rheological behavior—Langmuir, 2000, Vol. 16, No. 12, 5324-5332”;

“Stimuli responsive amphiphilic copolymers of sodium 2-(acrylamido)-2-methylpropanesulfonate and associative macromonomers—Polym. Preprint, Div. Polym. Chem. 1999, 40(2), 220-221”.

The ethylenically unsaturated hydrophobic monomers of these copolymers can be chosen from the acrylates or acrylamides of formula (IV) below:

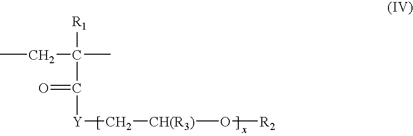

wherein R₁ and R₃, which may be identical or different, are chosen from a hydrogen atom and linear or branched C₁-C₆ alkyl radicals (such as methyl); Y is chosen from O and NH; R₂ is chosen from hydrophobic hydrocarbon-based radicals containing at least from 8 to 50 carbon atoms, for example, from 8 to 22 carbon atoms, and further as an example, from 6 to 18 carbon atoms such as from 12 to 18 carbon atoms; x is chosen from a number of moles of alkylene oxide and ranges from 0 to 100.

The radical R₂, for example, can be chosen from linear C₆-C₁₈ alkyl radicals (for example, n-hexyl, n-octyl, n-decyl, n-hexadecyl and n-dodecyl) and branched or cyclic C₆-C₁₈ alkyl radicals (for example, cyclododecane (C₁₂) or adamantane (C₁₀)); C₆-C₁₈ perfluoroalkyl radicals (for example the group of formula —(CH₂)₂—(CF₂)_{g}—CF₃); the cholesteryl radical (C₂₇) or a cholesterol ester residue, for instance the cholesteryl oxyhexanoate group; aromatic polycyclic groups, for instance naphthalene or pyrene. For instance, R₂ is chosen from linear alkyl radicals such as the n-dodecyl radical.

In certain embodiments, the monomer of formula (IV) comprises at least one alkylene oxide unit (x≧1) such as a polyoxyalkylenated chain. The polyoxyalkylenated chain may be constituted of ethylene oxide units and/or of propylene oxide units, for instance, constituted of ethylene oxide units. The number of oxyalkylene units may range from 3 to 100, for example, from 3 to 50, and further as an example, from 7 to 25.

Among these polymers, non-limiting mention may be made of:

crosslinked or non-crosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer, such as those described in European Patent Application Publication No. EP-A-750 899;

terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Non-limiting mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Additional non-limiting mention may be made of the copolymers constituted of 2-acrylamido-2-methylpropanesulphonic acid (AMPS) units of formula (V) below:

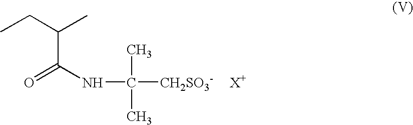

wherein X⁺ is chosen from protons, alkali metal cations, alkaline-earth metal cations, and the ammonium ion;
and of units of formula (VI) below:

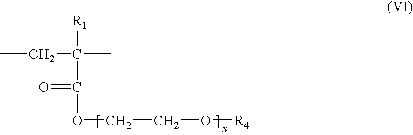

wherein x is chosen from an integer ranging from 3 to 100, for example, from 5 to 80 such as from 7 to 25; R₁ has the same meaning as that given above in formula (IV) and R₄ is chosen from linear or branched C₆-C₂₂ alkyls such as linear or branched C₁₀-C₂₂ alkyls.

Further as non-limiting examples, the polymers can be chosen from those with x=25, R₁ is chosen from methyl and R₄ is chosen from n-dodecyl; they are described in the Morishima articles mentioned above.

Additional non-limiting examples include the polymers wherein X₊ is chosen from sodium and ammonium.

(iii) The cationic amphiphilic polymers used in the present disclosure can be chosen from polyacrylates containing amine side groups.

The polyacrylates containing quaternized or non-quaternized amine side groups contain, for example, hydrophobic groups of the type such as steareth-20 (polyoxyethylenated (20) stearyl alcohol) or (C₁₀-C₃₀)alkyl PEG-20 itaconate.

Examples of polyacrylates containing amino side chains that may be mentioned are the polymers 8781-124B or 9492-103 or STRUCTURE PLUS from the company National Starch.

(iv) As amphoteric amphiphilic polymers containing at least one fatty chain, non-limiting mention may be made of copolymers of methacrylamidopropyltrimethylammonium chloride/acrylic acid/C₁₀-C₃₀ alkyl methacrylate, the alkyl radical can be, for example, a stearyl radical.
(b) Among the crosslinked acrylic acid homopolymers that may be mentioned are those crosslinked with an allyl alcohol ether of the sugar series, such as, for example, the products sold under the names CARBOPOL 980, 981, 954, 2984 and 5984 by the company Goodrich or the products sold under the names SYNTHALEN M and SYNTHALEN K by the company 3 VSA.
(c) Among the crosslinked copolymers of (meth)acrylic acid and of C₁-C₆ alkyl acrylate that may be mentioned is the product sold under the name VISCOATEX 538C by the company Coatex, which is a crosslinked copolymer of methacrylic acid and of ethyl acrylate as an aqueous dispersion containing 38% active material, or the product sold under the name ACULYN 33 by the company Rohm & Haas, which is a crosslinked copolymer of acrylic acid and of ethyl acrylate as an aqueous dispersion containing 28% active material. Non-limiting mention may also be made of the crosslinked methacrylic acid/ethyl acrylate copolymer in the form of an aqueous 30% dispersion manufactured and sold under the name CARBOPOL AQUA SF-1 by the company Noveon.
(d) Among the non-ionic homopolymers or copolymers containing ethylenically unsaturated monomers of ester and/or amide type, non-limiting mention may be made of the products sold under the names: CYANAMER P250 by the company Cytec (polyacrylamide); PMMA MBX-8C by the company US Cosmetics (methyl methacrylate/ethylene glycol dimethacrylate copolymer); ACRYLOID B66 by the company Rohm & Haas (butyl methacrylate/methyl methacrylate copolymer); BPA 500 by the company Kobo (polymethyl methacrylate).
(e) Among the ammonium acrylate homopolymers that may be mentioned, in a non-limiting manner, is the product sold under the name MICROSAP PAS 5193 by the company Hoechst.

Among the copolymers of ammonium acrylate and of acrylamide that may be mentioned, in a non-limiting manner, is the product sold under the name BOZEPOL C NOUVEAU or the product PAS 5193 sold by the company Hoechst. These products are described and prepared in documents French Patent Document FR-2 416 723, and U.S. Pat. Nos. 2,798,053 and 2,923,692.
(f) Poly(meth)acrylamido(C₁-C₄)alkylsulphonic acids

In some embodiments, the poly(meth)acrylamido(C₁-C₄)alkylsulphonic acid(s) is (are) can be crosslinked.

In some embodiments, the poly(meth)acrylamido(C₁-C₄)alkylsulphonic acid(s) is (are) partially or totally neutralized.

In some embodiments, the poly(meth)acrylamido(C₁-C₄)alkylsulphonic acid(s) is (are) water-soluble or water-swellable polymers.

Among these polymers, non-limiting mention may be made of:
polyacrylamidomethanesulphonic acid,
polyacrylamidoethanesulphonic acid,
polyacrylamidopropanesulphonic acid,
poly-(2-acrylamido-2-methylpropanesulphonic acid),
poly-(2-methylacrylamido-2-methylpropanesulphonic acid),
poly-(2-acrylamido-n-butanesulphonic acid).

Polymers of this type and crosslinked and partially or totally neutralized poly-(2-acrylamido-2-methylpropanesulphonic acid)s are known, described and prepared in document Germany Patent Publication No. DE-196 25 810.

These polymers may comprise, randomly distributed:
a) from 90% to 99.9% by weight of units of formula (VII) below:

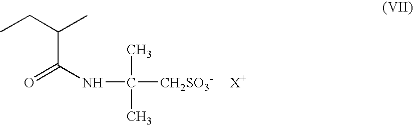

wherein X⁺ is chosen from a cation or a mixture of cations, including H⁺,
b) from 0.01% to 10% by weight of at least one crosslinking unit comprising at least two olefinic double bonds,

the weight proportions being defined relative to the total weight of the polymer;

X⁺ is chosen from a cation or a mixture of cations, for instance, is chosen from protons, alkali metal cations, cations equivalent to that of alkaline-earth metals, and an ammonium ion.

The crosslinked and neutralized poly-(2-acrylamido-2-methylpropanesulphonic acid) may comprise from 98% to 99.5% by weight of units of formula (VII) and from 0.5% to 2% by weight of crosslinking units.

The crosslinking units containing at least two olefinic double bonds are chosen, for example, from dipropylene glycol diallyl ether, polyglycol diallyl ethers, triethylene glycol divinyl ether, hydroquinone diallyl ether, tetraallyloxyethane, other poly-functional alcohol allyl or vinyl ethers, tetraethylene glycol diacrylate, triallylamine, trimethylolpropane diallyl ether, methylene-bisacrylamide, and divinylbenzene.

The crosslinking units comprising at least two olefinic double bonds can be chosen from those corresponding to the general formula (VIII) below:

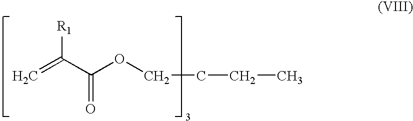

wherein R₁ is chosen from a hydrogen atom and C₁-C₄ alkyls such as methyl (trimethylolpropane triacrylate).

The crosslinked and partially or totally neutralized poly(2-acrylamido-2-methylpropanesulphonic acid)s are known under the names “Ammonium polyacrylamido-2-methylpropanesulphonate” or “Ammonium polyacryldimethyltauramide” (INCI name).

A product that may be used according to the present disclosure includes but not limited to the one sold by the company Clariant under the trade name HOSTACERIN AMPS; this is a crosslinked poly(2-acrylamido-2-methylpropanesulphonic acid) partially neutralized with aqueous ammonia.
(g) The crosslinked polymers of methacryloyloxy(C₁-C₄)alkyltri(C₁-C₄)alkylammonium salts such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound such as methylenebisacrylamide. A crosslinked acrylamide/methacryloyloxy-ethyltrimethylammonium chloride copolymer (20/80 by weight) in the form of a dispersion containing 50% by weight of said copolymer in mineral oil may be used. This dispersion is sold under the name “SALCARE® SC 92” by the company Ciba. A crosslinked methacryloyloxyethyltrimethylammoniurn chloride homopolymer containing about 50% by weight of the homopolymer in mineral oil or in a liquid ester may also be used. These dispersions are sold under the names “SALCARE® SC 95” and “SALCARE® SC 96” by the company Ciba.

As a non-limiting example, the (meth)acrylic thickening polymer(s) according to the present disclosure is (are) anionic.

The composition according to the present disclosure may comprise from 0.05 to 20%, such as from 0.1 to 10% by weight of at least one (meth)acrylic thickening polymers relative to the total weight of the composition.

In some embodiments, the cosmetically acceptable medium is aqueous.

The cosmetic composition according to the present disclosure may also comprise at least one organic solvent, for example, in an amount ranging from 0.05 to 40%, such as from 1 to 20% by weight, relative to the total weight of the composition.

This organic solvent may be chosen from C₂ to C₄ lower alcohols, for instance, ethanol, polyols and polyol ethers such as propylene glycol, polyethylene glycol or glycerol.

The compositions according to the present disclosure may also contain other cosmetically acceptable adjuvants, non-limiting examples of which include: ionic or non-ionic surfactants, additional thickeners other than the (meth)acrylic thickening polymers used in the compositions according to the present application, ethoxylated or non-ethoxylated fatty alcohols, co-thickeners, penetrants, fragrances, dyes, plasticizers, buffers, and various customary adjuvants such as waxes, volatile or non-volatile silicones that are cyclic or linear or branched, and are organomodified by alkoxylated or modified by amine groups or are unmodified, for example silicone gums, ceramides, pseudoceramides, plant, mineral or synthetic oils, vitamins or provitamins such as panthenol, opacifiers, reducing agents, emulsifiers, preservatives, mineral fillers, pearlescent agents, flakes, sunscreens, proteins, anionic, non-ionic, cationic or amphoteric fixing polymers, moisturisers, emollients, demulcents, anti-foaming agents, antiperspirants, free-radical scavengers, bactericides, sequestrants, anti-dandruff agents, antioxidants, basifying agents, acidifying agents, and any other additive conventionally used in cosmetic compositions intended to be applied to the hair.

The surfactants that can be used in the composition according to the present disclosure may be chosen from anionic, non-ionic, amphoteric, and cationic surfactants, and mixtures thereof.

Among the anionic surfactants that can be used, alone or as mixtures, in the context of the present disclosure, non-limiting mention may be made of salts, for example, alkali metal salts such as sodium salts, ammonium salts, amine salts, amino alcohol salts or alkaline-earth metal salts, for example magnesium salts, of the following compounds: alkyl sulphates, alkyl ether sulphates, alkylamido ether sulphates, alkylarylpolyether sulphates, monoglyceride sulphates; alkylsulphonates, alkylamidesulphonates, alkylaryl-sulphonates, α-olefin sulphonates, paraffin sulphonates; alkylsulphosuccinates, alkyl ether sulphosuccinates, alkylamide sulphosuccinates; alkylsulphoacetates; acylsarcosinates; and acylglutamates, the alkyl and acyl groups of all these compounds containing from 6 to 24 carbon atoms and the aryl group can be chosen from, for instance, phenyl and benzyl groups.

In some embodiments, it is also possible to use C₆-C₂₄ alkyl esters of polyglycoside carboxylic acids such as alkyl glucoside citrates, polyalkyl glycoside tartrates, and polyalkyl glycoside sulphosuccinates; alkylsulphosuccinamates, acylisethionates and N-acyltaurates, the alkyl or acyl group of all these compounds containing from 12 to 20 carbon atoms. Among the anionic surfactants that may also be used, non-limiting mention may also be made of acyllactylates in which the acyl group contains from 8 to 20 carbon atoms.

In addition, non-limiting mention may also be made of alkyl-D-galactosideuronic acids and the salts thereof, and also polyoxyalkylenated (C₆-C₂₄)alkyl ether carboxylic acids, polyoxyalkylenated (C₆-C₂₄)alkyl(C₆-C₂₄)aryl ether carboxylic acids, polyoxyalkylenated (C₆-C₂₄)alkylamido ether carboxylic acids and salts thereof, for example, those containing from 2 to 50 ethylene oxide groups, and mixtures thereof.

The non-ionic surfactants that may be used in the context of the present disclosure include but not limited to, compounds that are known (see, for instance, in “Handbook of Surfactants” by M. R. Porter, published by Blackie & Son (Glasgow and London), 1991, pp. 116-178). In some embodiments, they can be chosen from polyethoxylated, polypropoxylated or polyglycerolated fatty acids, (C₁-C₂₀)alkylphenols, a-diols or alcohols having a fatty chain containing, for example, 8 to 18 carbon atoms, it being possible for the number of ethylene oxide or propylene oxide groups to range from 2 to 50 and for the number of glycerol groups to range from 2 to 30. Non-limiting mention may also be made of copolymers of ethylene oxide and of propylene oxide, condensates of ethylene oxide and of propylene oxide with fatty alcohols; polyethoxylated fatty amides having, for example, from 2 to 30 mol of ethylene oxide, polyglycerolated fatty amides containing on average 1 to 5, such as 1.5 to 4, glycerol groups; polyethoxylated fatty amines having, for example, 2 to 30 mol of ethylene oxide; ethoxylated fatty acid esters of sorbitan having from 2 to 30 mol of ethylene oxide; fatty acid esters of sucrose, fatty acid esters of polyethylene glycol, (C₅-C₂₄)alkylpolyglucosides, N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

The amphoteric surfactants that are suitable for use in the present disclosure may include but not limited to: aliphatic secondary or tertiary amine derivatives, wherein the aliphatic group is chosen from linear or branched chains containing 8 to 22 carbon atoms and containing at least one water-solubilizing anionic group, such as, for example, a carboxylate, sulphonate, sulphate, phosphate or phosphonate group; non-limiting mention may also be made of (C₈-C₂₀)alkylbetaines, sulphobetaines, (C₈-C₂₀)alkylamido(C₆-C₈)-alkylbetaines or (C₈-C₂₀)alkylamido(C₆-C₈)alkylsulphobetaines; and mixtures thereof.

Among the amine derivatives that may be mentioned are the products sold under the name MIRANOL®, as described in U.S. Pat. No. 2,528,378 and U.S. Pat. No. 2,781,354 and classified in the CTFA dictionary, 3rd edition, 1982, under the names amphocarboxyglycinate and amphocarboxypropionate, and having the respective structures (1) and (2):

R₂—CONHCH₂CH₂—N⁺(R₃)(R₄)(CH₂COO⁻) (1)

wherein:
R₂ is chosen from alkyl groups derived from an acid R₂—COOH present in hydrolysed coconut oil, a heptyl, nonyl, and undecyl group,
R₃ is chosen from a β-hydroxyethyl group, and
R₄ is chosen from a carboxymethyl group; and

R₂—CONHCH₂CH₂—N(B)(C) (2)

wherein:
B is chosen from —CH₂CH₂OX′,
C is chosen from —(CH₂)_{z}—Y, with z=1 or 2,
X′ is chosen from the —CH₂CH₂—COOH group and a hydrogen atom,
Y′ is chosen from —COOH and the —CH₂—CHOH—SO₃H group,
R₂ is chosen from the alkyl groups of an acid R₂′—COOH present in coconut oil or in hydrolysed linseed oil, alkyl groups such as a C₁₇ group and its iso form, and unsaturated C₁₇ groups.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of non-limiting example, mention may be made of the cocoamphodiacetate sold under the trade name MIRANOL® C2M concentrate by the company Rhodia.

Among the amphoteric surfactants that may be used include, but not limited to, (C₈-C₂₀)alkylbetaines such as cocobetaine, (C₈-C₂₀)alkylamido(C₆-C₈)alkylbetaines such as cocamidobetaine, and alkylamphodiacetates, for instance disodium cocoamphodiacetate, and mixtures thereof.

The composition according to the present disclosure may also comprise at least one cationic surfactants that are known, such as primary, secondary or tertiary fatty amine salts, optionally polyoxyalkylenated; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

The non-ionic, amphoteric and cationic surfactants described above may be used alone or as mixtures and present in an amount ranging from 0.01% to 30% by weight, for example, ranging from 0.05% to 20% by weight such as ranging from 0.1% to 10% by weight, relative to the total weight of the composition.

The additional gelling agents and/or thickeners other than the (meth)acrylic thickening polymers suitable for the compositions of the present disclosure may be known in the art and may be chosen from poly(oxyalkylene) glycols, poly(oxyalkylene) glycol esters, alginates, biosaccharides, starch derivatives, natural gums such as xanthan gun, guar gum, carob bean gum, scleroglucans, derivatives of chitin and of chitosan, carrageenans, clays, and mixtures thereof.

By way of non-limiting example, gelling agents that are in the aqueous phase, mention may be made of FUCOGEL® 1000 PP sold by the company Solabia, SATIAGEL® KSO sold by Degussa and KELTROL® sold by the company Kelco.

The additional gelling agents can be present in an amount ranging from 0.05 to 15%, for example from 0.5 to 10% by weight of the composition.

The silicones that may be used as additives in the cosmetic compositions of the present disclosure can be chosen from volatile or non-volatile, cyclic, linear or branched silicones, optionally modified with organic groups, having a viscosity from 5×10⁻⁶ to 2.5 m²/s at 25° C., such as from 1×10⁻⁵ to 1 m²/s.

The silicones that can be used in accordance with the present disclosure may be soluble or insoluble in the composition and for instance, may be polyorganosiloxanes that are insoluble in the composition of the present disclosure. They may be in the form of oils, waxes, resins or gums.

The organopolysiloxanes are defined in Walter Noll's “Chemistry and Technology of Silicones” (1968), Academic Press. They can be volatile or non-volatile.

When they are volatile, the silicones may be chosen from those having a boiling point ranging from 60° C. to 260° C., and may also be chosen from:

(i) cyclic silicones comprising from 3 to 7, such as from 4 to 5 silicon atoms. These are, for example, octamethylcyclotetrasiloxane sold under the name VOLATILE SILICONE® 7207 by Union Carbide or SILBIONE® 70045 V2 by Rhodia, decamethylcyclopentasiloxane sold under the name VOLATILE SILICONE® 7158 by Union Carbide, and SILBIONE® 70045 V5 by Rhodia, and mixtures thereof.

Non-limiting mention may also be made of cyclocopolymers of the dimethylsiloxane/methylalkylsiloxane type, such as SILICONE VOLATILE® FZ 3109 sold by the company Union Carbide, of formula:

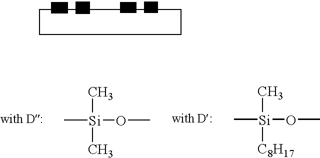

Non-limiting mention may also be made of mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones comprising 2 to 9 silicon atoms and having a viscosity of less than or equal to 5×10⁻⁶ m²/s at 25° C. A non-limiting example is decamethyltetrasiloxane sold under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, such as polyalkylsiloxanes, polyarylsiloxanes, polyalkylarylsiloxanes, silicone gums and resins, polyorganosiloxanes modified with organofunctional groups, and mixtures thereof, can be used.

These silicones may be chosen from polyalkylsiloxanes, among which non-limiting mention may be made of polydimethylsiloxanes containing trimethylsilyl end groups. The viscosity of the silicones is measured at 25° C. according to ASTM 445 standard Appendix C.

Among these polyalkylsiloxanes, mention may be made, in a non-limiting manner, of the following commercial products:
the SILBIONE® oils of the 47 and 70 047 series or the MIRASIL® oils sold by Rhodia, such as, for example, the oil 70 047 V 500 000;
the oils of the MIRASIL® series sold by the company Rhodia;
the oils of the 200 series from the company Dow Corning, such as, DC200 with a viscosity of 60 000 mm²/s;
the VISCASIL® oils from General Electric and certain oils of the SF series (SF 96, SF 18) from General Electric.

Non-limiting mention may also be made of polymethylsiloxanes containing dimethylsilanol end groups, known by the name dimethiconol (CTFA), such as the oils of the 48 series from the company Rhodia.

In this category of polyalkylsiloxanes, non-limiting mention may also be made of the products sold under the names ABIL WAX® 9800 and 9801 by the company Goldschmidt, which are poly(C₁-C₂₀)alkylsiloxanes.

The polyalkylarylsiloxanes may be chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, non-limiting mention may be made, by way of example, of the products sold under the following names:

the SILBIONE® oils of the 70 641 series from Rhodia;

the oils of the RHODORSIL® 70 633 and 763 series from Rhodia;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

The silicone gums that can be used in accordance with the present disclosure may be chosen from polyorganosiloxanes having high number-average molecular weights ranging from 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Non-limiting mention may be made of the following products:
polydimethylsiloxane gums,
polydimethylsiloxane/methylvinylsiloxane gums,
polydimethylsiloxane/diphenylsiloxane gums,
polydimethylsiloxane/phenylmethylsiloxane gums, and
polydimethylsiloxane/diphenylsilaxane/methylvinylsiloxane gums.

Products that can be used in accordance with the disclosure include but not limited to mixtures such as:

mixtures formed from a polydimethylsiloxane hydroxylated at the end of the chain, or dimethiconol (CTFA) and from a cyclic polydimethylsiloxane also called cyclomethicone (CTFA), such as the product Q2 1401 sold by the company Dow Corning;

mixtures formed from a polydimethylsiloxane gum with a cyclic silicone, such as the product SF 1214 SILICONE FLUID from the company General Electric; this product is an SF 30 gum corresponding to a dimethicone, having a number-average molecular weight of 500 000, dissolved in the oil SF 1202 SILICONE FLUID corresponding to decamethylcyclopentasiloxane;

mixtures of two PDMSs of different viscosities, for instance, of a PDMS gum and a PDMS oil, such as the product SF 1236 from the company General Electric. The product SF 1236 is the mixture of an SE 30 gum defined above, having a viscosity of 20 m²/s, and an SF 96 oil, with a viscosity of 5×10⁻⁶ m²/s. This product may contain 15% SE 30 gum and 85% SF 96 oil.

The organopolysiloxane resins that can be used in accordance with the present disclosure are crosslinked siloxane systems containing the following units:

R₂SiO_{2/2}, R₃SiO_{1/2}, RSiO_{3/2} and SiO_{4/2}

wherein R is chosen from hydrocarbon-based groups containing 1 to 16 carbon atoms and a phenyl group. Among these products, non-limiting examples include the ones wherein R is chosen from C₁-C₄ lower alkyl groups such as methyl and a phenyl group.

Among these resins, mention may be made of the product sold under the name Dow Corning 593 or those sold under the names SILICONE FLUID SS 4230 and SS 4267 by the company General Electric, which are silicones of dimethyl/trimethylsiloxane structure.

Mention may also be made of the trimethylsiloxysilicate type resins sold under the names X22-4914, X21-5034 and X21-5037 by the company Shin-Etsu.

The organomodified silicones that can be used in accordance with the present disclosure further include silicones as defined above and containing in their structure at least one organofunctional groups attached via a hydrocarbon-based group.

Among the organomodified silicones, non-limiting mention may be made of polyorganosiloxanes comprising:
polyethyleneoxy and/or polypropyleneoxy groups optionally containing C₆-C₂₄ alkyl groups, such as the products known as dimethicone copolyol sold by the company Dow Corning under the name DC 1248 or the oils SILWET® L 722, L 7500, L 77, L 711 from the company Union Carbide and the (C₁₂)alkylmethicone copolyol sold by the company Dow Corning under the name Q2 5200;
substituted or unsubstituted amine groups, such as the products sold under the name GP 4 SILICONE FLUID and GP 7100 by the company Genesee, or the products sold under the names Q2 8220 and Dow Corning 929 or 939 or Dow Corning 2-8299 by the company Dow Corning or the product sold under the name BELSIL ADM LOG 1 by the company Wacker. The substituted amine groups may be chosen from, for example, C₁-C₄ aminoalkyl groups;
thiol groups such as the products sold under the names GP 72A and GP 71 from Genesee;
alkoxylated groups such as the product sold under the name SILICONE COPOLYMER F-755 by SWS Silicones and ABIL WAX® 2428, 2434 and 2440 by the company Goldschmidt;
hydroxylated groups such as the polyorganosiloxanes containing a hydroxyalkyl functional group;
alkoxyalkyl groups such as, for example, the polyorganosiloxanes described in U.S. Pat. No. 4,957,732;
anionic groups of carboxylic type, such as, for example, in the products described in European Patent Publication No. EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names ABIL® S201 and ABIL® S255;
hydroxyacrylamino groups, such as the polyorganosiloxanes described in European Patent Publication No. EP 342 834. Non-limiting mention may be made, for example, of the product Q2-8413 from the company Dow Corning.

The silicones as described above may be used, alone or as a mixture, in an amount ranging from 0.01% to 20% by weight, for example, ranging from 0.1% to 5% by weight.

The compositions of the present disclosure may also comprise non-silicone fatty substances chosen from mineral, plant, animal and synthetic oils, waxes, fatty esters, ethoxylated or non-ethoxylated fatty alcohols, and fatty acids.

As oils that can be used in the composition of the present disclosure, non-limiting examples that may be mentioned include:
hydrocarbon-based oils of animal origin, such as perhydrosqualene;
hydrocarbon-based oils of plant origin, such as liquid fatty acid triglycerides containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stéarineries Dubois or those sold under the names MIGLYOL® 810, 812 and 818 by the company Dynamit Nobel, jojoba oil and shea butter oil;
linear or branched hydrocarbons of mineral or synthetic origin, such as volatile or non-volatile liquid paraffins, and derivatives thereof, petroleum jelly, polydecenes, hydrogenated polyisobutene such as PARLEAM®; isoparaffins, for instance isohexadecane and isodecane;
partially hydrocarbon-based and/or silicone-based fluoro oils, for instance those described in document Japanese Patent No. 2-295 912; fluoro oils that may also be mentioned include perfluoromethylcyclopentane and perfluoro-1,3-dimethylcyclohexane, sold under the names FLUTEC® PC1 and FLUTEC® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name FORALKYL® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The wax(es) may be chosen from, for example, carnauba wax, candelilla wax, esparto grass wax, paraffin wax, ozokerite, plant waxes such as olive wax, rice wax, hydrogenated jojoba wax or the absolute waxes of flowers such as the essential wax of blackcurrant blossom sold by the company Bertin (France), animal waxes, for instance beeswaxes or modified beeswaxes (cerabellina); other waxes or waxy starting materials that can be used according to the present disclosure include marine waxes such as the product sold by the company Sophim under the reference M82, and polyethylene waxes or polyolefin waxes in general.

The saturated or unsaturated fatty acids may be chosen from myristic acid, palmitic acid, stearic acid, behenic acid, oleic acid, linoleic acid, linolenic acid and isostearic acid.

The fatty esters may be chosen from carboxylic acid esters, for instance, mono-, di-, tri-, or tetracarboxylic esters.

The carboxylic acid esters can be chosen from esters of saturated or unsaturated, linear or branched C1-C26 aliphatic acids, and of saturated or unsaturated, linear or branched C1-C26 aliphatic alcohols, the total carbon number of the esters being greater than or equal to 10.

Among the monoesters, non-limiting mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

The following esters may also be used: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate and neopentyl glycol diheptanoate. The esters mentioned above being different from the esters of formula (I).

In some embodiments, the esters are chosen from ethyl palmitate, isopropyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

As fatty alcohols, mention may be made of linear or branched, saturated or unsaturated fatty alcohols containing from 8 to 26 carbon atoms, for instance cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol.

The fatty substances can be present in an amount ranging from 0.1 to 50%; such as from 1 to 30%, and for example, from 2 to 20% by weight of the total composition.

As indicated previously, the compositions may further comprise fixing polymers other than the polymers used according to the present disclosure. The term “fixing polymer” is understood within the meaning of the present disclosure to mean any polymer that makes it possible to give a shape to the hair or to hold the hair in a given shape.

All the anionic, cationic, amphoteric and non-ionic fixing polymers and mixtures thereof used in the art may be used in the compositions according to the present application.

The fixing polymers may be soluble in the cosmetically acceptable medium or insoluble in this same medium and used in this case in the form of dispersions of solid or liquid particles of polymer (latex or pseudolatex).

The anionic fixing polymers may be chosen from polymers comprising groups derived from carboxylic, sulphonic or phosphoric acid and have a number-average molecular weight ranging from 500 to 5 000 000.

In some embodiments, the anionic fixing polymers containing carboxylic groups may be chosen from:

A) The copolymers of acrylic acid and of acrylamide sold in the form of their sodium salts under the names RETEN 421, 423 or 425 by the company Hercules, the sodium salts of polyhydroxycarboxylic acids.

B) Copolymers of acrylic or methacrylic acid with a monoethylenic monomer, such as ethylene, styrene, vinyl esters, acrylic or methacrylic acid esters, optionally grafted onto a polyalkylene glycol such as polyethylene glycol and optionally crosslinked. Such polymers are described, for instance, in French Patent No. 1 222 944 and German Patent Application No. 2 330 956, the copolymers of this type comprising an optionally N-alkylated and/or hydroxyalkylated acrylamide unit in their chain as described in Luxembourg Patent Application Nos. 75370 and 75371 or sold under the name QUADRAMER by the company American Cyanamid. Non-limiting mention may also be made of the acrylic acid/ethyl acrylate/N-tert-butylacrylamide terpolymers such as ULTRAHOLD STRONG sold by the company BASF. Non-limiting mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name ACRYLIDONE® LM and methacrylic acid/ethyl acrylate/tert-butyl acrylate terpolymers, such as the product sold under the name LUVIMER® 100 P by the company BASF.

Non-limiting mention may also be made of methacrylic acid/acrylic acid/ethyl acrylate/methyl methacrylate copolymers as an aqueous dispersion, sold under the name AMERHOLD® DR 25 by the company Amerchol.

C) Crotonic acid copolymers, such as those comprising vinyl acetate or propionate units in their chain and optionally other monomers, such as allyl esters or methallyl esters, vinyl ether or vinyl ester of a linear or branched saturated carboxylic acid with a long hydrocarbon chain such as those containing at least 5 carbon atoms, it being possible for these polymers optionally to be grafted or crosslinked by another vinyl, allyl or methallyl ester monomer of an α- or β-cyclic carboxylic acid. Such polymers are described, inter alia, in French Patent Nos. 1 222 944, 1 580 545, 2 265 782, 2 265 781, 1 564 110 and 2 439 798. Commercial products falling into this class are the resins 28-29-30, 26-13-14 and 28-13-10 sold by the company National Starch.

D) Copolymers of C₄-C₈ monounsaturated carboxylic acids or anhydrides chosen from:
copolymers comprising (i) at least one maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described in, for example, U.S. Pat. Nos. 2,047,398, 2,723,248 and 2,102,113 and United Kingdom Patent No. GB 839 805. Commercial products include those sold under the names GANTREZ® AN or ES by the company ISP,
copolymers comprising (i) at least one maleic, citraconic or itaconic anhydride units and (ii) at least one monomers chosen from allyl or methallyl esters optionally comprising at least one acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

These polymers are described, for example, in French Patent Nos. 2 350 384 and 2 357 241 by the Applicant.

E) Polyacrylamides comprising carboxylate groups.

F) Homopolymers and copolymers comprising sulphonic groups such as polymers comprising vinylsulphonic, styrenesulphonic, naphthalenesulphonic or acrylamidoalkylsulphonic units, different from the branched sulphonic polyesters used according to the present disclosure.

These polymers can be chosen from, for example:
polyvinylsulphonic acid salts having a molecular weight of approximately ranging from 1000 to 100 000, and also the copolymers with an unsaturated comonomer such as acrylic or methacrylic acids and their esters, and also acrylamide or its derivatives, vinyl ethers and vinylpyrrolidone;
polystyrenesulphonic acid salts such as the sodium salts that are sold for example under the names FLEXAN® 500 and FLEXAN® 130 by National Starch. These compounds are described in French Patent No. FR 2 198 719;
polyacrylamidesulphonic acid salts, such as those mentioned in U.S. Pat. No. 4,128,631 and for example, polyacrylamidoethylpropanesulphonic acid sold under the name COSMEDIA POLYMER HSP 1180 by Henkel.

As another anionic fixing polymer that can be used according to the present disclosure, non-limiting mention may be made of the branched block anionic polymer sold under the name FIXATE G-100 by the company Lubrizol.

According to the present disclosure, the anionic fixing polymers can be chosen from copolymers of acrylic acid or of acrylic esters, such as the acrylic acid/ethyl acrylate/N-tert-butylacrylamide terpolymers sold, for instance, under the name ULTRAHOLD® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold, for example, under the name RESIN 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name GANTREZ® by the company ISP, the copolymers of methacrylic acid and of methyl methacrylate sold under the name EUDRAGIT® L by the company Rohm Pharma, the copolymers of methacrylic acid and of ethyl acrylate sold under the name LUVIMER® MAEX or MAE by the company BASF, the vinyl acetate/crotonic acid copolymers sold under the name LUVISET CA 66 by the company BASF, the vinyl acetate/crotonic acid copolymers grafted with polyethylene glycol sold under the name ARISTOFLEX® A by the company BASF, and the polymer sold under the name FIXATE G-100 L by the company Lubrizol.

The cationic fixing film-forming polymers that can be used according to the present disclosure can be chosen from polymers comprising primary, secondary, tertiary and/or quaternary amine groups forming part of the polymer chain or directly attached thereto, and having a molecular weight ranging from 500 to 5 000 000 such as from 1 000 to 3 000 000.

Among these polymers, non-limiting mention may be made of the following cationic polymers:

(1) homopolymers or copolymers derived from acrylic or methacrylic esters or amides and comprising at least one of the units of the following formulae:

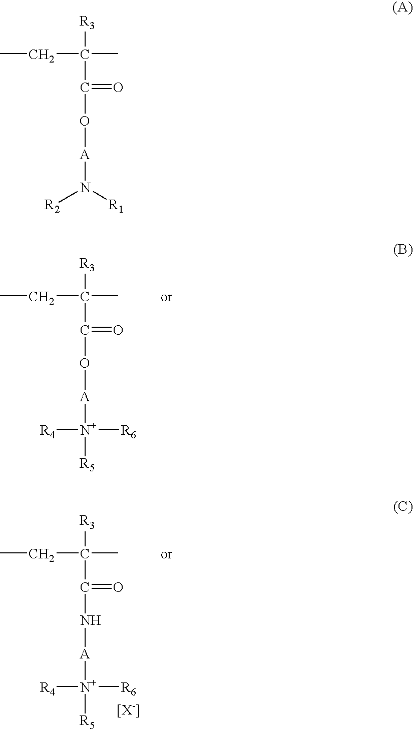

wherein:

R₃ is chosen from a hydrogen atom and a CH₃ radical;

A is chosen from linear or branched alkyl groups comprising 1 to 6 carbon atoms or hydroxyalkyl groups comprising 1 to 4 carbon atoms;

R₄, R₅ and R₆, which may be identical or different, are chosen from alkyl groups having from 1 to 18 carbon atoms and a benzyl radical;

R₁ and R₂, which may be identical or different, are chosen from a hydrogen atom and alkyl groups having from 1 to 6 carbon atoms;

X is chosen from a methosulphate anion and halides such as chloride or bromide.

The copolymers of the family (1) also comprise at least one unit derived from comonomers that may be chosen from the family of acrylamides, methacrylamides, diacetone acrylamides, acrylamides and methacrylamides substituted on the nitrogen with lower (C₁₋₄) alkyl groups, groups derived from acrylic or methacrylic acids or esters thereof, vinyllactams such as vinylpyrrolidone or vinylcaprolactam, and vinyl esters.

Thus, among these copolymers of the family (1), non-limiting mention may be made of:
copolymers of acrylamide and of dimethylaminoethyl methacrylate quaternized with dimethyl sulphate or with a dimethyl halide, such as the product sold under the name HERCOFLOC® by the company Hercules,
copolymers of acrylamide and of methacryloyloxyethyltrimethylammonium chloride, described, for example, in European Patent Application No. EP-A-080 976 and sold under the name BINA QUAT P 100 by the company Ciba Geigy,
copolymers of acrylamide and of methacryloyloxyethyltrimethylammonium methosulphate, such as the product sold under the name RETEN by the company Hercules,
quaternized or non-quaternized vinylpyrrolidone/dialkylaminoalkyl acrylate or methacrylate copolymers, such as the products sold under the name “GAFQUAT®” by the company ISP, such as, for example, “GAFQUAT® 734” or “GAFQUAT® 755”, or alternatively the products known as “COPOLYMER® 845, 958 and 937”. These polymers are described in detail in French Patent Nos. 2 077 143 and 2 393 573,
fatty-chain polymers containing a vinylpyrrolidone unit, such as the products sold under the name STYLEZE W20 and STYLEZE W10 by the company ISP,
dimethylaminoethyl methacrylate/vinylcaprolactam/vinylpyrrolidone terpolymers, such as the product sold under the name GAFFIX VC 713 by the company ISP, and
quaternized vinylpyrrolidone/dimethylaminopropylmethacrylamide copolymers, such as the products sold under the name “GAFQUAT® HS 100” by the company ISP;

(2) non-cellulosic cationic polysaccharides, for instance, the ones containing quaternary ammonium, such as those described in U.S. Pat. Nos. 3,589,578 and 4 031 307, such as guar gums containing trialkylammonium cationic groups. Such products are sold, for example, under the trade names JAGUAR C13S, JAGUAR C 15 and JAGUAR C 17 by the company Meyhall;

(3) quaternary copolymers of vinylpyrrolidone and of vinylimidazole;

(4) chitosans or salts thereof; the salts that can be used are, for instance, chitosan acetate, lactate, glutamate, gluconate or pyrrolidonecarboxylate.

Among these compounds, non-limiting mention may be made of chitosan having a degree of deacetylation of 90.5% by weight, sold under the name KYTAN BRUT Standard by the company Aber Technologies, and chitosan pyrrolidonecarboxylate sold under the name KYTAMER® PC by the company Amerchol.

(5) cationic cellulose derivatives such as copolymers of cellulose or of cellulose derivatives grafted with a water-soluble monomer comprising a quaternary ammonium, and disclosed in, for example, U.S. Pat. No. 4,131,576, such as hydroxyalkylcelluloses, for instance, hydroxymethyl-, hydroxyethyl- or hydroxypropylcelluloses grafted with, for example, a methacryloyloxyethyltrimethylammonium, methacrylamidopropyltrimethylammonium or dimethyldiallylammonium salt.

The products sold corresponding to this definition are, for example, the products sold under the name “CELQUAT L 200” and “CELQUAT H 100” by the company National Starch.

The amphoteric fixing polymers that can be used in accordance with the present disclosure can be chosen from polymers comprising units B and C distributed randomly in the polymer chain, wherein B is chosen from units derived from a monomer comprising at least one basic nitrogen atom and C is chosen from units derived from an acid monomer comprising at least one carboxylic or sulphonic groups, or alternatively B and C can be chosen from groups derived from carboxybetaine or sulphobetaine zwitterionic monomers;

B and C can also be chosen from cationic polymer chains comprising primary, secondary, tertiary or quaternary amine groups, wherein at least one of the amine groups bears a carboxylic or sulphonic group connected via a hydrocarbon group or alternatively B and C form part of a chain of a polymer containing an α,β-dicarboxylic ethylene unit in which one of the carboxylic groups has been made to react with a polyamine comprising at least one primary or secondary amine groups.

The amphoteric fixing polymers corresponding to the definition given above may include but not limited to the following polymers:

(1) copolymers having acidic vinyl units and basic vinyl units, such as those resulting from the copolymerization of a monomer derived from a vinyl compound bearing a carboxylic group such as acrylic acid, methacrylic acid, maleic acid, or α-chloroacrylic acid, and a basic monomer derived from a substituted vinyl compound containing at least one basic atom, such as dialkylaminoalkyl methacrylates and acrylates, dialkylaminoalkylmethacrylamides and -acrylamides. Such compounds are described in U.S. Pat. No. 3,836,537.

(2) polymers comprising units derived from:
a) at least one monomer chosen from acrylamides and methacrylamides substituted on the nitrogen atom with an alkyl group,
b) at least one acidic comonomer containing at least one reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides that may be used in according to the present disclosure include, for instance, compounds in which the alkyl groups comprise from 2 to 12 carbon atoms, for example, N-ethylacrylamide, N-tert-butyl-acrylamide, N-tert-octylacrylamide, N-octylacrylamide, N-decylacrylamide, N-dodecylacryl-amide and the corresponding methacrylamides.

The acidic comonomers are chosen from, for example, acrylic acid, methacrylic acid, crotonic acid, itaconic acid, maleic acid and fumaric acid and alkyl monoesters, having 1 to 4 carbon atoms, of maleic or fumaric acids or anhydrides.

In some embodiments, the basic comonomers are chosen from aminoethyl, butylaminoethyl, N,N′-dimethylaminoethyl and N-tert-butylaminoethyl methacrylates.

The copolymers whose CTFA (4th edition, 1991) name is octylacryl-amide/acrylates/butylaminoethyl methacrylate copolymer, such as the products sold under the name AMPHOMER® or LOVOCRYL® 47 by the company National Starch, can be used.

(3) crosslinked and acylated polyamino amides partially or totally derived from polyamino amides of general formula:

—[CO—R₁₀—CO—Z—] (IX)

wherein R₁₀ is chosen from divalent groups derived from a saturated dicarboxylic acid, a mono- or dicarboxylic aliphatic acid containing an ethylenic double bond, an ester of a lower alkanol, having 1 to 6 carbon atoms, of these acids, and groups derived from the addition of any one of said acids to a bis(primary) or bis(secondary) amine, and Z is chosen from groups derived from a bis(primary), mono- or bis(secondary) polyalkylene-polyamine comprised of:

a) in an amount ranging from 60 to 100 mol % of the group:

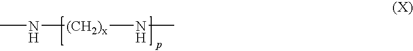

wherein x=2 and p=2 or 3, or alternatively x=3 and p=2
this group being derived from the groups chosen from diethylenetriamine, triethylenetetraamine, and dipropylenetriamine;

b) in an amount ranging from 0 to 40 mol % of the group (X) above, wherein x=2 and p=1 and which is derived from the group chosen from ethylenediamine and the groups derived from piperazine:

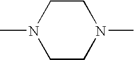

c) in an amount ranging from 0 to 20 mol %, of the —NH—(CH₂)₆—NH— group being derived from hexamethylenediamine,

these polyamino amides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids can be chosen from acids having from 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation may be chosen from propane sultone and butane sultone; the salts of the acylating agents can be chosen from, for example, sodium and potassium salts.

(4) polymers comprising zwitterionic units of formula:

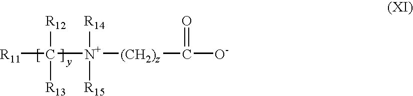

wherein R₁₁ is chosen from polymerizable unsaturated groups such as an acrylate, methacrylate, acrylamide or methacrylamide group, y and z are chosen from an integer ranging from 1 to 3, R₁₂ and R₁₃ are chosen from a hydrogen atom, methyl, ethyl, and propyl groups, R₁₄ and R₁₅ are chosen from a hydrogen atom and alkyl groups such that the sum of the carbon atoms in R₁₄ and R₁₅ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.

By way of example, mention may be made of the copolymers of methyl methacrylate/methyl dimethylcarboxymethylammonioethyl methacrylate such as the product sold under the name DIAFORMER Z301 by the company Sandoz.

(5) polymers derived from chitosan comprising monomer units corresponding to the following formulae:

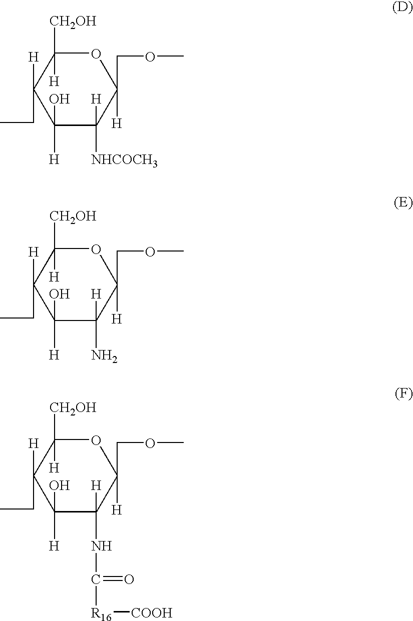

the unit (D) being present in an amount ranging from 0 to 30%, the unit (E) being present in an amount ranging from 5% to 50% and the unit (F) being present in an amount ranging from 30% and 90%, it being understood that, in this unit (F), R₁₆ is chosen from a group of formula:

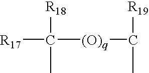

wherein, if q=0, R₁₇, R₁₈ and R₁₉, which may be identical or different, are chosen from hydrogen atom, methyl, hydroxyl, acetoxy, amino residues, monoalkylamine residues, dialkylamine residues that are optionally interrupted by at least one nitrogen atom and/or optionally substituted with at least one group chosen from amine, hydroxyl, carboxyl, alkylthio, sulphonic groups, and alkylthio residues wherein the alkyl group bears an amino residue, at least one of the groups R₁₇, R₁₈ and R₁₉ being, in this case, a hydrogen atom;

or, if q=1, R₁₇, R₁₈ and R_{1g} are hydrogen atoms, as well as the salts formed by these compounds with bases or acids.

(6) polymers with units corresponding to the general formula (XII) are described, for example, in French Patent No. 1 400 366:

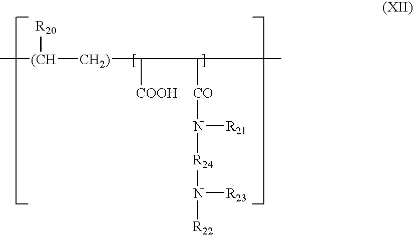

wherein R₂₀ is chosen from a hydrogen atom, CH₃O, CH₃CH₂O, and phenyl group, R₂₁ is chosen from a hydrogen atom and lower alkyl groups such as methyl or ethyl, R₂₂ is chosen from a hydrogen atom and C₁₋₆ lower alkyl groups such as methyl or ethyl, R₂₃ is chosen from C₁₋₆ lower alkyl groups such as methyl or ethyl or a group corresponding to the formula: —R₂₄—N(R₂₂)₂, R₂₄ is chosen from —CH₂—CH₂—, —CH₂—CH₂—CH₂— or —CH₂—CH(CH₃)— group, R₂₂ having the meanings mentioned above.

(7) polymers derived from the N-carboxyalkylation of chitosan, such as N-carboxymethylchitosan or N-carboxybutylchitosan sold under the name “EVALSAN” by the company Jan Dekker.

(8) amphoteric polymers of the type -D-X-D-X chosen from:
a) polymers obtained by the action of chloroacetic acid or sodium chloroacetate on compounds comprising at least one unit of formula:

-D-X-D-X-D- (XIII)

where D denotes a group

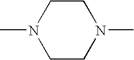

and X is chosen from the symbol E or E′. E or E′, which may be identical or different, are chosen from divalent groups that are alkylene groups with a straight or branched chain containing up to 7 carbon atoms in the main chain, which is unsubstituted or substituted with hydroxyl groups and which can comprise, in addition to oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;

b) polymers of formula:

-D-X-D-X— (XIV)

where D is chosen from a group

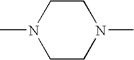

and X is chosen from the symbols E and E′ and at least one E′; E having the meaning given above and E′ is a divalent group that is chosen from alkylene groups with a straight or branched chain having up to 7 carbon atoms in the main chain, which is unsubstituted or substituted with at least one hydroxyl groups and containing at least one nitrogen atom, the nitrogen atom being substituted with an alkyl chain that is optionally interrupted by an oxygen atom and comprising at least one carboxyl function or at least one hydroxyl function and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric fixing polymers mentioned above, non-limiting examples include but not limited to those of family (3), such as the copolymers whose CTFA name is octylacrylamide/acrylates/butylaminoethyl methacrylate copolymer, such as the products sold under the names AMPHOMER®, AMPHOMER® LV 71 or LOVOCRYI® 47 by the company National Starch and those of family (4) such as the copolymers of methyl methacrylate/methyl dimethylcarboxymethylammonioethyl methacrylate, sold, for example, under the name DIAFORMER Z301 by the company Sandoz.

The non-ionic fixing polymers that may be used according to the present disclosure are chosen, for example, from:
polyalkyloxazolines;
vinyl acetate homopolymers;
vinyl acetate copolymers, for instance copolymers of vinyl acetate and of acrylic ester, copolymers of vinyl acetate and of ethylene, or copolymers of vinyl acetate and of maleic ester, for example of dibutyl maleate;
homopolymers and copolymers of acrylic esters, for instance copolymers of alkyl acrylates and of alkyl methacrylates, such as the products sold by the company Rohm & Haas under the names PRIMAL® AC-261 K and EUDRAGIT® NE 30 D, by the company BASF under the name 8845, or by the company Hoechst under the name APPRETAN® N9212;
copolymers of acrylonitrile and of a non-ionic monomer chosen, for example, from butadiene and alkyl (meth)acrylates; mention may be made of the products sold under the name CJ 0601 B by the company Rohm & Haas;
styrene homopolymers;
styrene copolymers, for instance copolymers of styrene and of an alkyl (meth)acrylate, such as the products MOWILITH® LDM 6911, MOWILITH® DM 611 and MOWILITH® LDM 6070 sold by the company Hoechst, and the products RHODOPAS® SD 215 and RHODOPAS® DS 910 sold by the company Rhone-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers such as vinylpyrrolidone homopolymers and such as the polyvinylcaprolactam sold under the name LUVISKOL® Plus by the company BASF; and
vinyllactam copolymers such as a poly(vinylpyrrolidone/vinyllactam) copolymer sold under the trade name LUVITEC® VPC 55K65W by the company BASF, poly(vinylpyrrolidone/vinyl acetate) copolymers, such as those sold under the name PVPVA® S630L by the company ISP, LUVISKOL® VA 73, VA 64, VA 55, VA 37 and VA 28 by the company BASF; and poly(vinylpyrrolidone/vinyl acetate/vinyl propionate) terpolymers, for instance the product sold under the name LUVISKOL® VAP 343 by the company BASF.

The alkyl groups of the non-ionic polymers mentioned above may contain from 1 to 6 carbon atoms.

According to the present disclosure, it is also possible to use fixing polymers of grafted silicone type comprising a polysiloxane portion and a portion comprising a non-silicone organic chain, one of the two portions constituting the main chain of the polymer, and the other being grafted onto said main chain.

These polymers are described, for example, in European Patent Application Publication Nos. EP-A-0 412 704, EP-A-0 412 707, EP-A-0 640 105, and EP-A-0 582 152, and International Patent Publication Nos. WO 95/00578, and WO 93/23009 and U.S. Pat. Nos. 4,693,935, 4,728,571, and 4 972 037.

These polymers may be chosen from amphoteric, anionic, and non-ionic, such as anionic or non-ionic.

Such polymers are, for example, copolymers that can be obtained by free radical polymerization from the monomer mixture formed from:

a) an amount ranging from 50 to 90% by weight of tert-butyl acrylate;

b) an amount ranging from 0 to 40% by weight of acrylic acid;

c) an amount ranging from 5 to 40% by weight of a silicone macromer of formula:

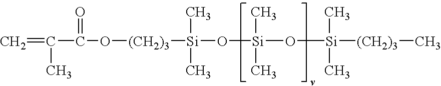

wherein v is a number ranging from 5 to 700, the weight percentages being calculated relative to the total weight of the monomers.

Other examples of grafted silicone polymers include but not limited to, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Another type of silicone fixing polymer that may be mentioned is the product LUVIFLEX® Silk, sold by the company BASF.

Functionalized or non-functionalized, silicone or non-silicone, cationic, non-ionic, anionic or amphoteric polyurethanes or mixtures thereof may also be used as fixing polymers.

In some embodiments, the polyurethanes include those disclosed in European Patent Application Publication Nos. EP 0 751 162, EP 0 637 600, and EP 0 648 485, and French Patent Publication No. 2 743 297, of which the Applicant is the Proprietor, and European Patent Application No. EP 0 656 021 and International Patent Publication No. WO 94/03510 from the company BASF and European Patent Publication No. EP 0 619 111 from the company National Starch.

Polyurethanes that may be used for the present disclosure include but not limited to, the products sold under the names LUVISET Pur® and LUVISET® Si Pur by the company BASF.

The concentration of fixing polymer(s) used in the compositions according to the present disclosure can be in an amount ranging from 0.1% and 20% such as ranging from 0.5% and 10% by weight relative to the total weight of the composition.

A person skilled in the art should know how to add the additives without disturbing the properties of the compositions of the present disclosure.

In some embodiments, the compositions are in the form of gels. For instance, the compositions have a viscosity greater than 500 cps at a temperature of 25° C. and at a shear rate of 1 s-1.

When the composition according to the present disclosure is packaged in an aerosol device, it further comprises at least one propellant, which may be chosen from volatile hydrocarbons, such as N-butane, propane, isobutane, pentane, halogenated hydrocarbons and mixes thereof. It is also possible to use carbon dioxide, nitrous oxide, dimethyl ether (DME), nitrogen or compressed air as the propellant. Mixtures of propellants can also be used. For example, dimethyl ether is used.

The propellant may be present at a concentration ranging from 5 to 90% by weight relative to the total weight of the composition in the aerosol device and, for example, at a concentration ranging from 10% to 60%.

The composition according to the disclosure may be used as a leave-in application on the hair.

Another aspect of the present disclosure includes a method for shaping the hair, comprising the application of a cosmetic composition according to the present disclosure. In some embodiments, the present disclosure relates to a styling method comprising the application of a composition according to the present disclosure to the hair, optional rinsing of the hair, then the shaping and drying of the hair.

The examples that follow illustrate the disclosure without limiting the scope thereof.

### EXAMPLES

The following compositions were produced:

The concentrations are expressed as grams of active material per 100 g of composition.

### Examples of Gels

| | Compositions | | | |
|---|---|---|---|---|
| | A | B | C | D |
| Branched sulphonic | 1 | 2 | 3 | 3.8 |
| polyester (1) | | | | |
| VP/VA copolymer (2) | 3 | — | — | — |
| Acrylates/C10-C30 | 1.65 | — | — | — |
| alkyl acrylate | | | | |
| crosslinked polymer (3) | | | | |
| Glyceryl | — | — | — | 0.15 |
| polyacrylate (4) | | | | |
| Hydroxypropylated | — | 0.35 | — | — |
| guar gum | | | | |
| Carbopol 980 (5) | — | 0.17 | — | — |
| Polyacryloyl | — | — | 1.5 | |
| dimethyl | | | | |
| ammonium taurate (6) | | | | |
| Polyacrylate-3 (7) | — | — | — | 0.8 |
| AMP-acrylates/allyl | — | 2.8 | — | — |
| methacrylates | | | | |
| copolymer (8) | | | | |
| VP/dimethyl- | 1.5 | — | — | — |
| aminoethyl | | | | |
| methacrylate | | | | |
| copolymer (9) | | | | |
| Fragrance | 0.4 | 0.4 | 0.5 | 0.5 |
| PEG-40 | 0.8 | 0.8 | 1 | 1 |
| hydrogenated | | | | |
| castor oil (10) | | | | |
| Pentasodium | — | 0.05 | 0.1 | — |
| pentetate | | | | |
| Triethanolamine | 2 | 1.5 | — | 0.22 |
| Preservative | 1 | 0.95 | 0.35 | 0.95 |
| Sorbitol | — | — | 2.7 | — |
| PEG-8 | — | — | — | 2.2 |
| Glycerol | 3 | — | 1.8 | 1.8 |
| Propylene glycol | 3 | 3 | 2.5 | — |
| Denatured alcohol | 2.8 | — | — | — |
| Water | qs for 100 | qs for 100 | qs for 100 | qs for 100 |

(1) Eastman AQ 1350 ®
(2) vinylpyrrolidone/vinyl acetate copolymer LUVISKOL ® VA 64P from BASF
(3) PEMULEN TR-1 (Lubrizol)
(4) HISPAGEL 200 (Cognis)
(5) crosslinked polyacrylic acid sold by Lubrizol
(6) HOSTACERIN AMPS (Clariant)
(7) VISCOPHOBE DB1000 (Dow Chemical)
(8) FIXATE G100L (Lubrizol)
(9) Copolymere 845 (ISP)
(10) CREMOPHOR CO 40 (BASF)

The above compositions exhibited an adequate consistency that allowed an easy application and an easy positioning on clear hair. After drying, the hair exhibited a natural hold of long duration.

What is claimed is:

1. A cosmetic composition comprising, in a cosmetically acceptable medium:
(i) at least one branched sulphonic polyester;
(ii) at least one (meth)acrylic thickening polymer; and
(iii) at least one surfactant chosen from anionic, non-ionic, amphoteric, and cationic surfactants.

2. The cosmetic composition according to claim 1, wherein the at least one branched sulphonic polyester is obtained by polycondensation of:
(a) at least one dicarboxylic acid that does not bear a sulphonic functional group,
(b) at least one diol or a mixture comprising at least one diol and at least one diamine,
(c) at least one monomer comprising two identical or different reactive functional groups chosen from hydroxyl, amino, and carboxyl groups, and at least one sulphonic functional group, and
(d) at least one monomer comprising at least three identical or different reactive functional groups chosen from hydroxyl, amino, and carboxyl groups.

3. The cosmetic composition according to claim 2, wherein the at least one branched sulphonic polyester further comprises units (e) derived from monomers comprising two different reactive functional groups chosen from hydroxy carboxylic acids and amino carboxylic acids.

4. The cosmetic composition according to claim 1, wherein the at least one branched sulphonic polyesters is present in an amount ranging from 0.2 to 15% by weight, relative to the total weight of the composition.

5. The cosmetic composition according to claim 1, wherein the at least one branched sulphonic polyesters is present in an amount ranging from 0.5 to 10% by weight, relative to the total weight of the composition.

6. The cosmetic composition according to claim 1, wherein the at least one (meth)acrylic thickening polymer is chosen from:
acrylic associative thickeners;
crosslinked acrylic acid homopolymers;
crosslinked copolymers of (meth)acrylic acid and of a (C₁-C₆)alkyl acrylate;
non-ionic homopolymers and copolymers containing ethylenically unsaturated monomers of ester and/or amide type;
ammonium acrylate homopolymers;
copolymers of ammonium acrylate and acrylamide;
(meth)acrylamido(C₁-C₄)alkylsulphonic acid homopolymers and copolymers; and
crosslinked methacryloyl(C₁-C₄)alkyltri(C₁-C₄)alkylammonium homopolymers and copolymers.

7. The cosmetic composition according to claim 1, wherein the at least one (meth)acrylic thickening polymer is anionic.

8. The cosmetic composition according to claim 1, wherein the at least one (meth)acrylic thickening polymer is present in an amount ranging from 0.05 to 20% by weight, relative to the total weight of the composition.

9. The cosmetic composition according to claim 1, wherein the at least one (meth)acrylic thickening polymer is present in an amount ranging from 0.1 to 10% by weight, relative to the total weight of the composition.

10. The cosmetic composition according to claim 1, further comprising at least one gelling agent and/or at least one additional thickener chosen from poly(oxyalkylene) glycols, poly(oxyalkylene) glycol esters, alginates, biosaccharides, starch derivatives, natural gums such as xanthan gum, guar gum, carob bean gum, scleroglucans, derivatives of chitin and of chitosan, carrageenans, and clays.

11. The cosmetic composition according to claim 1, further comprising at least one compound chosen from silicones and non-silicone fatty substances.

12. The cosmetic composition according to claim 11, wherein the non-silicone fatty substances are chosen from mineral, plant, animal and synthetic oils, waxes, fatty esters, ethoxylated and non-ethoxylated fatty alcohols, and fatty acids.

13. The cosmetic composition according to claim 1, further comprising at least one fixing polymer chosen from anionic, non-ionic, amphoteric, and cationic fixing polymers.

14. A method for styling or shaping keratin materials comprising applying the cosmetic composition according to claim 1 to the keratin materials.

15. The method according to claim 14, wherein the keratin materials are chosen from human keratin materials.

16. A method for styling or shaping hair comprising:
(1) lying the cosmetic composition according to claim 1 to the hair;
(2) optional rinsing the hair; and
(3) shaping and drying the hair.
